(19) United States
(12) Patent Application Publication
(10) Pub. No.: US 2019/0307368 A1
(43) Pub. Date: Oct. 10, 2019

(54) THERMOELECTRIC GENERATORS AND APPLICATIONS THEREOF

(71) Applicant: INTERNATIONAL THERMODYNE, INC., Charlotte, NC (US)

(72) Inventors: Katherine Holman HUGHES, Huntersville, NC (US); Timothy Alan RISSER, Charlotte, NC (US); John Paul SOLITARIO, Charlotte, NC (US); William David CHEMELEWSKI, Los Gatos, CA (US); Christopher Ned FOLMAR, Charlotte, NC (US)

(21) Appl. No.: 16/349,934

(22) PCT Filed: Nov. 14, 2017
(86) PCT No.: PCT/US17/61542

§ 371 Date: May 14, 2019

Related U.S. Application Data
(60) Provisional application No. 62/421,773, filed on Nov. 14, 2016.
(60) Provisional application No. 62/421,855, filed on Nov. 14, 2016.

(51) Int. Cl. A61B 5/11; G08C 17/02; A61B 5/00; H01L 35/02

(52) U.S. Cl. CPC A61B 5/1118; G08C 17/02; H02N 2/18; H01L 35/02; A61B 5/6804

(57) ABSTRACT

In one aspect, thermoelectric generators are described herein. A thermoelectric generator, in some embodiments, comprises a multilayer ribbon including an electrically insulating flexible substrate comprising a first side having a p-type material deposited thereon and a second side having an n-type material deposited thereon, wherein the p-type material and the n-type material are electrical communication. In another aspect, electronic systems for incorporation into apparel articles are described herein, which employ one or more energy harvesting mechanisms for powering various electronic functionalities. In some embodiments, for example, systems for monitoring apparel wear and/or use are provided. A system for monitoring apparel wear or use can comprise an energy harvesting component and a signaling component partially or fully powered by electrical energy generated by the energy harvesting component from wear or use of the apparel.

### RELATED APPLICATION DATA

The present invention claims priority pursuant to 35 U.S.C. § 119(e) to U.S. Provisional Patent Application Ser. Nos. 62/421,773 and 62/421,855 filed Nov. 14, 2016, each of which is incorporated herein by reference in its entirety.

### FIELD

The present invention relates to thermoelectric architectures and, in particular, to electrical generator apparatus incorporating thermoelectric materials.

### BACKGROUND

Thermoelectric materials and apparatus are widely used for the generation of electricity from heat sources. Thermoelectric apparatus, for example, can be employed to generate electricity from waste heat generated in various industrial applications. Thermoelectric efficiency is quantified by the Figure of Merit, ZT.

Thermoelectric materials demonstrating higher ZT values have higher thermoelectric efficiencies. Fabricating thermoelectric materials with reasonable ZT values is often difficult and/or expensive. Bismuth chalcogenides, for example, provide excellent thermoelectric properties with ZT values ranging from 0.7 to 1.0. These materials can be nanostructured to produce a superlattice structure of alternating Bi₂Te₃ and Bi₂Se₃ layers resulting in a material having acceptable electrical conductivity and poor thermal conductivity. Fabrication of these materials, nevertheless, can be time consuming and expensive.

Moreover, as a result of fabrication requirements and other material tolerances, many thermoelectric materials do not lend themselves to facile incorporation into a wide variety of devices for heat collection and electrical generation. The apparel industry, for example, is continuously looking for new features and designs that engage consumers and increase the functionality of clothing. Various smart fabrics and other textile compositions have been introduced operable to provide information to the wearer based on a physical change in the fabric, such as color or texture. Electronic functionalities have also been introduced into apparel articles. However, such apparel has exhibited limited success due to generally high costs and the requirement of battery power sources. Battery power sources are inherently incompatible with washing and drying of apparel articles. Moreover, batteries can be heavy and cumbersome and require replacement over time.

### SUMMARY

In one aspect, thermoelectric generators are described which, in some embodiments, can overcome or mitigate one or more disadvantages of current thermoelectric materials. As detailed further herein, architectures of these thermoelectric generators can facilitate their incorporation into a variety of environment for electrical generation, including incorporation into various apparel articles.

A thermoelectric generator, in some embodiments, comprises a multilayer ribbon including an electrically insulating flexible substrate comprising a first side having a p-type material deposited thereon and a second side having an n-type material deposited thereon, wherein the p-type material and the n-type material are in electrical communication. As described further herein, the p-type and n-type materials can be ink compositions including organic nanoparticles, inorganic nanoparticles or mixtures thereof. Further, the multilayer ribbon can exhibit a folded and/or rolled orientation to enhance output of the thermoelectric generator.

In another aspect, a thermoelectric generator comprises a multilayer ribbon including an electrically insulting flexible substrate comprising a first side having a first p-type material deposited thereon and a second side having a second p-type material deposited thereon, wherein the first and second p-type materials are in electrical communication. In other embodiments, a thermoelectric generator comprises a multilayer ribbon including an electrically insulting flexible substrate comprising a first side having a first n-type material deposited thereon and a second side having a second n-type material deposited thereon, wherein the first and second n-type materials are in electrical communication. As described further herein, the p-type and n-type materials can be ink compositions including organic nanoparticles, inorganic nanoparticles or mixtures thereof. Further, the multilayer ribbon can exhibit a folded and/or rolled orientation to enhance output of the thermoelectric generator.

In another aspect, methods of making thermoelectric generators are described herein. In some embodiments, a method of making a thermoelectric generator comprises providing a flexible ribbon substrate comprising a first side and a second side. A p-type material is deposited on the first side and an n-type material is deposited on the second side. The deposited p-type material and the n-type material are placed in electrical communication with one another. In some embodiments, the p-type material and n-type material are placed in electrical communication by cutting through the multilayer ribbon structure. Alternatively, an external electrical contact can extend from the p-type material to the n-type material. In other embodiments, a method of making a thermoelectric generator comprises providing a flexible ribbon substrate comprising a first side and a second sixe. A first p-type material is deposited on the first side and a second p-type material is deposited on the second side. The deposited first and second p-type materials are in electrical communication with one another. Alternatively, the first and second p-type materials can be replaced with first and second n-type materials to provide additional embodiments of thermoelectric generators.

In a further aspect, electric generators are described herein. An electric generator, in some embodiments, comprises a thermoelectric component positioned on a support. The thermoelectric component can comprise one or more thermoelectric generators exhibiting any architecture or properties described herein. In some embodiments, for example, a thermoelectric generator comprises a multilayer ribbon including p-type material and n-type material on opposing sides of an electrically insulating substrate. In other embodiments, the multilayer ribbon can employ first and second p-type materials or first and second n-type materials on opposing sides of the electrically insulating substrate. In further embodiments, a thermoelectric generator employs p-type material or n-type material on a single side of a substrate without p-type or n-type material on an opposing side of the substrate. In embodiments wherein a plurality of thermoelectric generators is present, the thermoelectric generators can be in electrical communication with one another. In some embodiments, thermoelectric generators in electrical communication with one another have the same construction. Alternatively, thermoelectric generators of differing construction and/or polarity can be in electrical communication with one another.

The thermoelectric component, in some embodiments, is covered by one or more protective layers. Additionally, the electric generator can further comprise a triboelectric component. The triboelectric component can include an electron donor layer in reversible contact with an electron acceptor layer. In some embodiments, the electron donor and electron acceptor layers are formed of differing polymeric materials.

In another aspect, electronic systems for incorporation into apparel articles are described herein, which employ one or more energy harvesting mechanisms for powering various electronic functionalities. In some embodiments, for example, systems for monitoring apparel wear and/or use are provided. A system for monitoring apparel wear or use can comprise an energy harvesting component and a signaling component partially or fully powered by electrical energy generated by the energy harvesting component from wear or use of the apparel. In some embodiments, the monitoring system further comprises an energy storage component for storing electrical energy generated by the energy harvesting component. In such embodiments, the energy storage component can participate in powering of the signaling component.

In another aspect, apparel compositions are described herein. An apparel composition, in some embodiments, comprises an article of apparel and a system coupled to the apparel article. The system comprises an energy harvesting component and a signaling component partially or fully powered by electrical energy generated by the energy harvesting component from wear or use of the apparel article. As described above, the system can further comprise an energy storage component for storing electrical energy generated by the energy harvesting component. The energy storage component can participate in powering of the signaling component.

In a further aspect, methods of monitoring use or wear of apparel are provided. In some embodiments, a method of monitoring use or wear of apparel comprises coupling a monitoring system to the apparel, the monitoring system comprising an energy harvesting component and a signaling component. Electrical energy is generated with the energy harvesting component from wear or use of the apparel. The electrical energy is employed to partially or fully power the signaling component to emit signal providing information regarding wear or use of the apparel. In some embodiments, the monitoring system further comprises an energy storage component for storing electrical energy generated by the energy harvesting component. Information regarding use or wear of the apparel, in some embodiments, comprises the time period over which the apparel is worn or the location of the apparel in a global positioning system. Information can also include temperature of the apparel.

These and other embodiments are described further in the following detailed description.

### BRIEF DESCRIPTION OF THE DRAWINGS

FIG. 1 illustrates an end view thermoelectric generator including a multilayer ribbon according to some embodiments described herein.

FIG. 2 illustrates a folded configuration of a multilayer ribbon according to some embodiments described herein.

FIG. 3 illustrates a wound configuration of a multilayer ribbon according to some embodiments described herein.

FIG. 4 illustrates a folded and wound configuration of a multilayer ribbon according to some embodiments described herein.

FIG. 5 illustrates a folded and wound configuration of a multilayer ribbon according to some embodiments described herein.

FIG. 6 illustrates a thermoelectric component of an electric generator according to some embodiments described herein.

FIG. 7 illustrates a thermoelectric component of an electric generator according to some embodiments described herein.

FIG. 8 illustrates an embodiment wherein thermoelectric generators of differing construction in electrical communication with one another.

FIG. 9 illustrates one embodiment where a thermoelectric generator comprising double-sided p-type material is in electrical communication with two thermoelectric generators comprising double-sided n-type materials.

FIG. 10 illustrates an electric generator according to some embodiments described herein.

FIG. 11 illustrates an electric generator comprising a thermoelectric component and triboelectric component according to some embodiments described herein.

FIG. 12 illustrates a cross-sectional view of thermoelectric generators according to some embodiments.

FIG. 13 illustrates a system for monitoring apparel wear or use according to some embodiments described herein.

FIG. 14 illustrates a system for monitoring apparel wear or use according to some embodiments described herein.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### I. Thermoelectric Generators

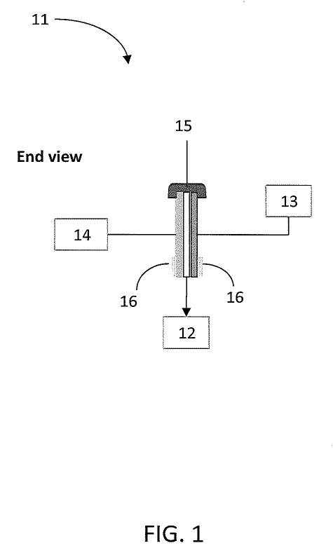

In one aspect, thermoelectric generators are described herein. A thermoelectric generator, in some embodiments, comprises a multilayer ribbon including an electrically insulating flexible substrate comprising a first side having a p-type material deposited thereon and a second side having an n-type material deposited thereon, wherein the p-type material and the n-type material are in electrical communication. FIG. 1 illustrates an end view of thermoelectric generator according to some embodiments described herein. As illustrated in FIG. 1, the multilayer ribbon 11 comprises an electrically insulating flexible substrate 12. A p-type layer 13 is positioned over a first side of the substrate 12, and an n-type layer 14 is positioned over a second side of the substrate 12. The p-type layer 13 and n-type layer 14 are in electrical communication via an electrical contact 15 extending from the p-type layer to the n-type layer. Additionally, conductive leads 16 can be placed on the p-type 13 and/or n-type 14 layers.

In another aspect, a thermoelectric generator comprises a multilayer ribbon including an electrically insulting flexible substrate comprising a first side having a first p-type material deposited thereon and a second side having a second p-type material deposited thereon, wherein the first and second p-type materials are in electrical communication. In other embodiments, a thermoelectric generator comprises a multilayer ribbon including an electrically insulting flexible substrate comprising a first side having a first n-type material deposited thereon and a second side having a second n-type material deposited thereon, wherein the first and second n-type materials are in electrical communication. Referring once again to FIG. 1, a first p-type layer 13 is positioned over a first side of the substrate 12, and a second p-type layer 14 is positioned over the second side of the substrate 12. The first and second p-type layers 13, 14 are in electrical communication via an electrical contact 15. Alternatively, a first n-type layer 13 is positioned over a first side of the substrate 12, and a second p-type layer 14 is positioned over the second side of the substrate 12.

Turning now to specific components, the electrically insulating flexible substrate can generally be formed of a polymeric material. Any polymeric material not inconsistent with the objectives of the present invention can be used in the production of an insulating substrate. In some embodiments, piezoelectric polymeric material is employed as the electrically insulating substrate. In such embodiments, the polymeric insulating substrate can comprise semicrystalline polymer including, but not limited to, polyvinyl fluoride (PVF), polyvinylidene fluoride (PVDF), polyvinylidene fluoride-trifluoroethylene (PVDF-TrFE), polyvinylidene fluoride-tetrafluoroethylene (PVDF-TFE), polytetrafluoroethylene (PTFE), or mixtures or copolymers thereof. Semicrystalline polymers of PVDF, PVDF-TFE and/or PVDF-TrFE used in insulating substrates of the thermoelectric apparatus can demonstrate increased amounts of β-phase. For example, PVDF, PVDF-TFE and/or PVDF-TrFE of an insulating layer can display a ratio of β/α of 1.5 to 2.5. In some embodiments, the β/α ratio is 2 to 2.5. As discussed herein, β-phase crystallites can be provided a non-random orientation by poling techniques, thereby enhancing piezoelectric and pyroelectric properties of the insulating layer.

A flexible insulating substrate, in some embodiments, comprises polyacrylic acid (PAA), polymethacrylate (PMA), polymethylmethacrylate (PMMA) or mixtures or copolymers thereof. In some embodiments, an insulating substrate comprises a polyolefin including, but not limited to polyethylene, polypropylene, polybutylene or mixtures or copolymers thereof.

A flexible insulating substrate can further comprise particles demonstrating piezoelectric behavior. For example, a polymeric insulating substrate can comprise particles of BaTiO₃, BiTe particles, other inorganic piezoelectric particles or mixtures thereof. The BaTiO₃ particles, BiTe particles and/or other inorganic particles can have any size and/or geometry not inconsistent with the objectives of the present invention. BaTiO₃ and BiTe particles can demonstrate a size distribution ranging from 20 nm to 500 nm. Further, piezoelectric particles can be dispersed in polymer of the insulation layer at any loading not inconsistent with the objectives of the present invention. In some embodiments, BaTiO₃ particles, BiTe particles and/or other inorganic piezoelectric particles are nanoparticles are present in an insulating substrate in an amount of 5-80 weight percent or 10-50 weight percent, based on the total weight of the insulating substrate. As described herein, piezoelectric particles of an insulating substrate can be electrically poled to further enhance the piezoelectric and/or pyroelectric properties of thermoelectric apparatus described herein.

Alternatively, an electrically insulating flexible substrate can be formed of an inorganic or ceramic material. In some embodiments, an insulating layer is formed of metal oxide particles, including transition metal oxide particles. Suitable metal oxide particles can also demonstrate piezoelectric behavior. In one embodiment, for example, an insulating substrate is formed of BaTiO₃ particles that can be electrically poled.

As described herein, a p-type material can be deposited on or coupled to a first side of the electrically insulating flexible substrate. In some embodiments, p-type material can be deposited on or coupled to both sides of the electrically insulating flexible substrate. Suitable p-type material can comprise organic materials, inorganic materials or various combinations thereof. In some embodiments, p-type material can include p-type organic nanoparticles, p-type inorganic nanoparticles or mixtures thereof. In some embodiments, p-type nanoparticles are selected from the group consisting of nanotubes, nanowires, nanorods, platelets and sheets. The p-type nanoparticles can have a 1-dimensional or 2-dimensional structure, in some embodiments.

P-type organic nanoparticles can include carbon nanotubes, fullerenes, graphene or mixtures thereof. In some embodiments, lattice structures of the organic p-type nanoparticles include one or more dopants such as boron. Alternatively, p-type dopant is externally applied to the organic nanoparticles by the environment surrounding the nanoparticles in a carrier or host material. For example, a polymeric material can provide p-dopant to surfaces of the organic nanoparticles. Similarly, one or more p-dopant species can be dispersed in a carrier for interaction with the organic nanoparticles. In some embodiments, p-dopant species in the carrier can include various salts, crown ethers or combinations thereof.

P-type inorganic nanoparticles can include binary, ternary and quaternary semiconductor compositions formed from elements selected from Groups IB, IIB and IIIA-VIA of the Periodic Table. For example, p-type inorganic nanoparticles can be formed of Cu₂₋ₓTe, Cu₂₋ₓSe, Sb₂Te₃, Ag₂Se, Ag₂Te, Cu₂Te, Cu₂Se, Se or Te. P-type inorganic nanoparticles can also be selected from various transition metal dichalcogenides, MX₂, where M is a transition metal and X is a chalcogen. In some embodiments, p-dopant is externally applied to inorganic nanoparticles by the environment surrounding the nanoparticles in a carrier or host material.

P-type material, in some embodiments, is deposited on one or more sides of the electrically insulating substrate as an ink composition. Organic and/or inorganic nanoparticles, for example, can be dispersed in a solvent to provide the ink. Suitable solvent systems can be chosen according to several considerations including compositional identity of the organic and/or inorganic nanoparticles, desired nanoparticle loading and compositional identities of other ink components including, but not limited to, polymeric component(s), surfactant and/or dopant. In some embodiments, water is a suitable solvent for the p-type ink composition.

Polymeric components of the ink composition can include fluoropolymers including polyvinylidene fluoride (PVDF), polyvinyl fluoride (PVF), polyvinylidene fluoride-trifluoroethylene (PVDF-TrFE), polyvinylidene fluoride-tetrafluoroethylene (PVDF-TFE), polytetrafluoroethylene (PTFE), or mixtures or copolymers thereof. In other embodiments, a polymeric component of the p-type ink can be semiconducting polymers. Suitable semiconducting polymers can include phenylene vinylenes, such as poly(phenylene vinylene) and poly(p-phenylene vinylene) (PPV), and derivatives thereof. In some embodiments, semiconducting polymers comprise poly fluorenes, naphthalenes, and derivatives thereof. In other embodiments, semiconducting polymers comprise 3,4-polyethylenedioxythiophene:polystyrenesulfonate (PEDOT:PSS), poly(2-vinylpyridine) (P2VP), polyamides, poly(N-vinylcarbazole) (PVCZ), polypyrrole (PPy), polyaniline (PAn) and poly[2,6-(4,4-bis-(2-ethylhexyl)-4H-cyclopenta[2,1-b;3,4-b′]dithiophene)-alt-4,7-(2,1,3-benzothiadiazole)] (PCPDTBT). In a further embodiment, a polymeric component of the ink can include polyethylene oxide or derivatives thereof.

Surfactant of the ink composition can be selected according to various considerations including chemical identity of the organic and/or inorganic nanoparticles. Suitable surfactant can include cationic surfactant, anionic surfactant, non-ionic surfactant or combinations thereof. In some embodiments, surfactant for carbon nanoparticles is selected from sodium dodecylbenzenesulfonate, sodium dodecyl sulfate, sodium cholate or Triton X-100.

Organic and/or inorganic nanoparticles can be present in the p-type ink composition in any desired amount. In some embodiments, organic and/or inorganic nanoparticles are present in an amount ranging from 1-80 weight percent. Nanoparticle loading in the ink can also be selected from values provided in Table I.

```
TABLE I
P-type Ink Nanoparticle Loading (wt. %)
  2-95
  5-60
10-60
15-50
20-60
```

The p-type ink can be deposited on the electrically insulating flexible substrate by a variety of techniques including, but not limited to, drop casting, air brushing, sonicated spray head printing, screen printing, slot die coating and roll printing.

In other embodiments, p-type material is provided as a preform, and the preform is coupled to one or more sides of the insulating flexible substrate. For example, a p-type preform can comprise a polymeric host containing organic and/or inorganic nanoparticles. The preform can be provided as a thin-polymeric film comprising organic and/or inorganic nanoparticles. Suitable preform architectures may resemble the p-type layers disclosed in PCT Patent Application PCT/US2011/056740 and/or PCT Patent Application PCT/US2014/027486, each of which is incorporated herein by reference in its entirety.

As described herein, an n-type material can be deposited on or coupled to a first side of the electrically insulating flexible substrate. In some embodiments, n-type material can be deposited on or coupled to both sides of the electrically insulating flexible substrate. N-type material can comprise organic materials, inorganic materials or various combinations thereof. In some embodiments, the n-type material can include n-type organic nanoparticles, n-type inorganic nanoparticles or mixtures thereof. In some embodiments, n-type nanoparticles are selected from the group consisting of nanotubes, nanowires, nanorods, platelets and sheets. The n-type nanoparticles can have a 1-dimensional or 2-dimensional structure, in some embodiments.

N-type organic nanoparticles can include carbon nanotubes, fullerenes, graphene or mixtures thereof. In some embodiments, lattice structures of the organic N-type nanoparticles include one or more dopants such as nitrogen. Alternatively, n-type dopant is externally applied to the organic nanoparticles by the environment surrounding the nanoparticles in a carrier or host material. For example, a polymeric material can provide n-dopant to surfaces of the organic nanoparticles. Similarly, one or more n-dopant species can be dispersed in a carrier for interaction with the organic nanoparticles. N-dopant species can include polyethyleneimine (PEI), crown ethers, n-DMBI and/or PEIE. Additional n-dopant species can include various transition metal complexes such as ruthenium pentamethylcyclopentadienyl mesitylene and the dimer of pentamethyl rhodocene.

N-type inorganic nanoparticles can include binary, ternary and quaternary semiconductors compositions formed from elements selected from Groups IB, IIB and IIIA-VIA of the Periodic Table. For example, n-type inorganic nanoparticles can be formed of Bi₂Se₃, Bi₂Te₃, Bi₂Te₃₋ₓSeₓ, Sb₂Te₃, Sb₂₋ₓBiₓTe₃, Cu doped Bi₂Se₃ and Ag surface modified Bi₂Se₃ and Bi₂Te₃. N-type inorganic nanoparticles can also be selected from various transition metal dichalcogenides, MX₂. In some embodiments, n-type transition metal dichalcogenides include TiS₂, WS₂ and MoS₂. In some embodiments, n-dopant is externally applied to inorganic nanoparticles by the environment surrounding the nanoparticles in a carrier or host material.

The n-type material can be deposited on the electrically insulating substrate as an ink composition. N-type organic and/or inorganic nanoparticles, for example, can be dispersed in a solvent to provide the ink. Suitable solvent systems can be chosen according to several considerations including compositional identity of the organic and/or inorganic nanoparticles, desired nanoparticle loading and compositional identities of other ink components including, but not limited to, polymeric component(s), surfactant and/or dopant. In some embodiments, water is a suitable solvent for the n-type ink composition.

Polymeric components of the n-type ink composition can include fluoropolymers including polyvinylidene fluoride (PVDF), polyvinyl fluoride (PVF), polyvinylidene fluoride-trifluoroethylene (PVDF-TrFE), polyvinylidene fluoride-tetrafluoroethylene (PVDF-TFE), polytetrafluoroethylene (PTFE), or mixtures or copolymers thereof. In other embodiments, a polymeric component of the n-type ink can be semiconducting polymers. Suitable semiconducting polymers can include phenylene vinylenes, such as poly(phenylene vinylene) and poly(p-phenylene vinylene) (PPV), and derivatives thereof. In some embodiments, semiconducting polymers comprise poly fluorenes, naphthalenes, and derivatives thereof. In other embodiments, semiconducting polymers comprise poly(2-vinylpyridine) (P2VP), polyamides, poly(N-vinylcarbazole) (PVCZ), polypyrrole (PPy), polyaniline (PAn) and poly[2,6-(4,4-bis-(2-ethylhexyl)-4H-cyclopenta[2,1-b;3,4-b′]dithiophene)-alt-4,7-(2,1,3-benzothiadiazole)] (PCPDTBT). In a further embodiment, a polymeric component of the ink can include polyethylene oxide or derivatives thereof.

Surfactant of the n-type ink composition can be selected according to various considerations including chemical identity of the organic and/or inorganic nanoparticles. Suitable surfactant can include cationic surfactant, anionic surfactant, non-ionic surfactant or combinations thereof. In some embodiments, surfactant for carbon nanoparticles is selected from sodium dodecylbenzenesulfonate, sodium dodecylsulfate, sodium cholate or Triton X-100.

Organic and/or inorganic nanoparticles can be present in the n-type ink composition in any desired amount. In some embodiments, organic and/or inorganic nanoparticles are present in an amount ranging from 1-80 weight percent. Nanoparticle loading in the n-type ink can also be selected from values provided in Table II.

```
TABLE II
N-type Ink Nanoparticle Loading (wt. %)
  2-95
  5-60
10-60
15-50
20-60
```

The n-type ink can be deposited on the electrically insulating flexible substrate by a variety of techniques including, but not limited to, drop casting, air brushing, sonicated spray head printing, screen printing, slot die coating and roll printing.

In other embodiments, the n-type material is provided as a preform, and the preform is coupled to one or more sides of the insulating flexible substrate. For example, an n-type preform can comprise a polymeric host containing organic and/or inorganic nanoparticles. The preform can be provided as a thin-polymeric film comprising organic and/or inorganic nanoparticles. Suitable preform architectures may resemble the n-type layers disclosed in PCT Patent Application PCT/US2011/056740 and/or PCT Patent Application PCT/US2014/027486.

The p-type material and the n-type material of the multilayer ribbon are in electrical communication. In some embodiments, the p-type material and n-type material are in direct contact with one another to establish the electrical communication. For example, the p-type material can extend from the first side and along an edge of the electrically insulating flexible substrate to contact the n-type material. Alternatively, the n-type material can extend from the first side and along an edge of the electrically insulating flexible substrate to contact the p-type material. In some embodiments, the multilayer ribbon can be cut where deformation of the ribbon at the cut location places the p-type and n-type materials in contact. An edge of multilayer may also be heat treated to bring the p-type and n-type materials into electrical communication by melting or partial melting.

As illustrated in FIG. 1, the p-type material and n-type material can also be in electrical communication via an electrical contact extending from the p-type material to the n-type material. In further embodiments, the electrical contact reinforces an architecture wherein the p-type material and n-type material are in direct contact with one another. The electrical contact can be any material suitable for establishing electrical communication between the p-type material and n-type material. In some embodiments, the electrical contact is formed of a metal or alloy. In other embodiments, the electrical contact can be carbon nanoparticles such as single-walled carbon nanotubes, multi-walled carbon nanotubes or graphene.

The foregoing architectures placing p-type material and n-type material in electrical communication can also be employed to place first and second p-type materials or first and second n-type materials in electrical communication.

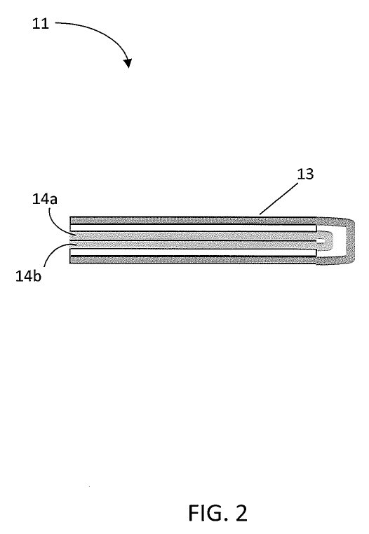

The multilayer ribbon can have any desired dimensions not inconsistent with the objectives of the present invention. In some embodiments, the multilayer ribbon is sufficiently wide to establish thermal gradients for thermoelectric behavior. For example, the multilayer ribbon can generally have width of 2-20 mm. Additionally, the multilayer ribbon can have any desired length. Referring now to FIG. 2, the multilayer ribbon can be folded back on itself such that contacting faces of p-type material or contacting faces of n-type material are formed. As illustrated in FIG. 2, the multilayer ribbon 11 is folded forming contacting faces 14(a), 14(b) of n-type material.

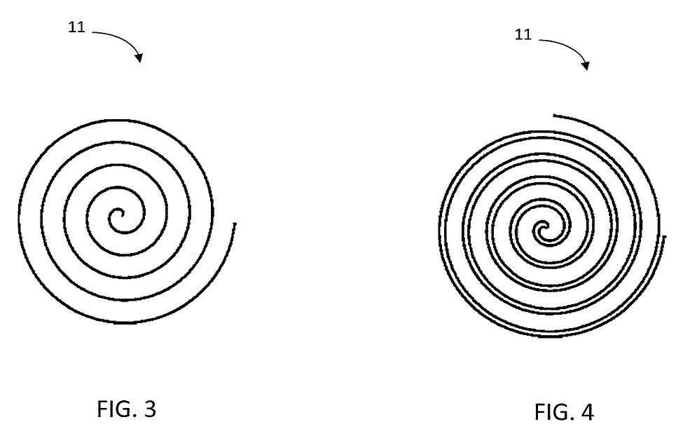

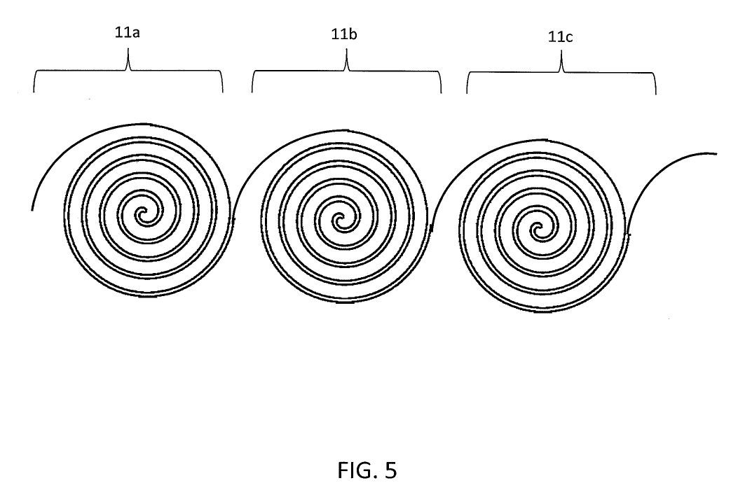

In further embodiments, the multilayer ribbon can have sufficient length to permit a wound configuration. FIG. 3 illustrates the multilayer ribbon 11 being placed into a wound configuration. In the embodiment of FIG. 3, the multilayer is wound into a spiral format. FIG. 4 also illustrates a wound or spiral format for the multilayer ribbon 11. In the embodiment of FIG. 4, however, the multilayer ribbon is folded as in FIG. 2 and then placed in the spiral format. Further, the multilayer ribbon can have sufficient length to permit winding into multiple spirals 11a, 11b and 11c as illustrated in FIG. 5. The foregoing configurations or orientations of the multilayer ribbon can permit tailoring of the power density provided by the thermoelectric generator.

Additionally, a plurality of thermoelectric generators having the ribbon architecture described herein can be arranged into a single device. For example, any number of the thermoelectric generators can be stacked and connected in series within the stack. In such embodiments, electrically insulating layers can be positioned between individual thermoelectric generators of the stack. In other embodiments, a plurality of thermoelectric generators of ribbon structure can be arranged in a lateral configuration and connected in series or parallel.

### II. Electric Generators

In a further aspect, electric generators are described herein. An electric generator, in some embodiments, comprises a thermoelectric component positioned on a support. The thermoelectric component can comprise one or more thermoelectric generators exhibiting any architecture or properties described herein. In some embodiments, for example, a thermoelectric generator comprises a multilayer ribbon including p-type material and n-type material on opposing sides of an electrically insulating substrate. In other embodiments, the multilayer ribbon can employ first and second p-type materials or first and second n-type materials on opposing sides of the electrically insulating substrate. In further embodiments, a thermoelectric generator employs p-type material or n-type material on a single side of a substrate without p-type or n-type material on an opposing side of the substrate. In embodiments wherein a plurality of thermoelectric generators is present, the thermoelectric generators can be in electrical communication with one another. In some embodiments, thermoelectric generators in electrical communication with one another have the same construction. Alternatively, thermoelectric generators of differing construction and/or polarity can be in electrical communication with one another.

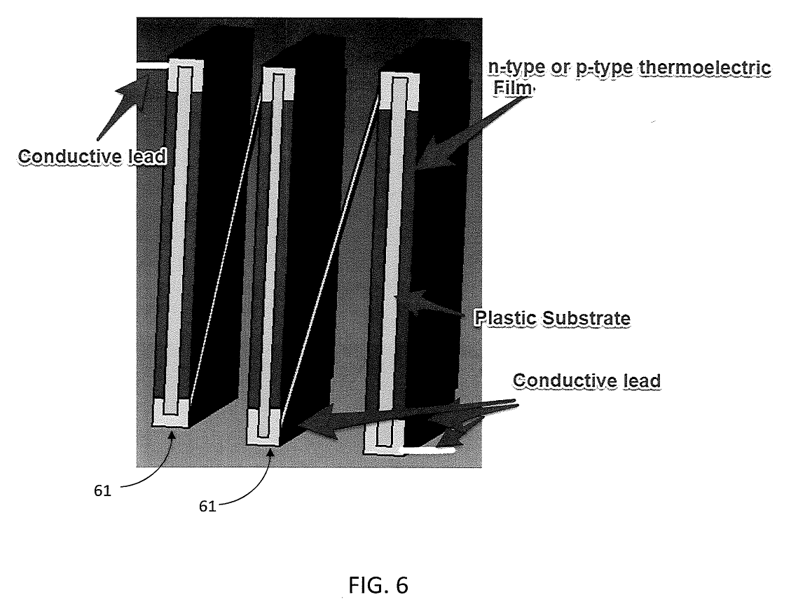

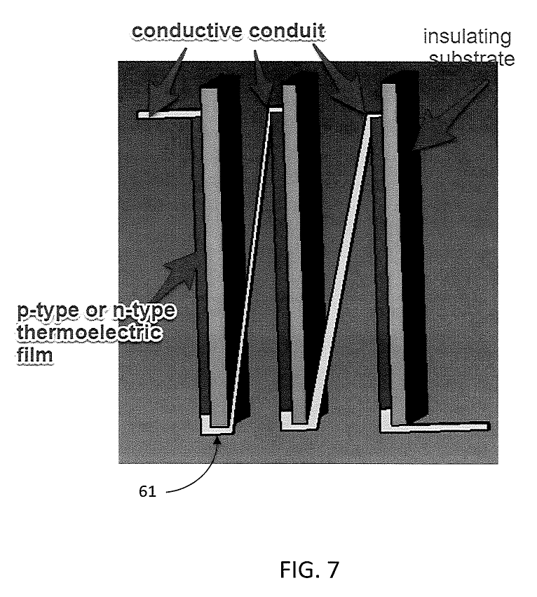

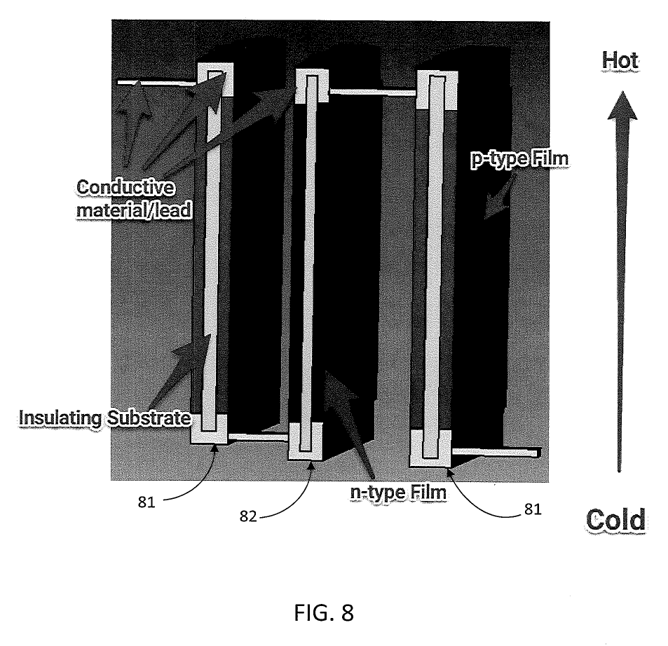

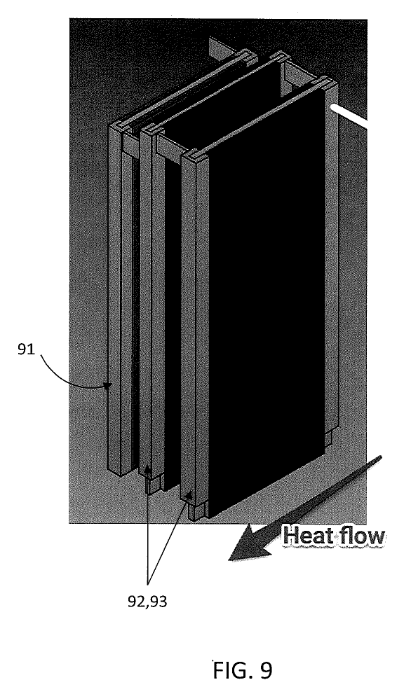

FIG. 6 illustrates a thermoelectric component of an electric generator according to some embodiments. In the embodiment of FIG. 6, each thermoelectric generator 61 comprises a multilayer ribbon wherein first and second p-type materials or first and second n-type materials are positioned on opposing sides of a substrate. Conductive leads place the thermoelectric generators in electrical communication. FIG. 7 illustrates a thermoelectric component of an electric generator according to other embodiments. The thermoelectric generators 71 of FIG. 7 employ p-type material or n-type material on a single side of the substrate without p-type or n-type material on opposing sides of the substrate. Conductive leads place the p-type or n-type materials in electrical communication. FIG. 8 illustrates an embodiment wherein thermoelectric generators of differing construction are in electrical communication with one another. In the embodiment of FIG. 8, thermoelectric generators 81 comprising double-sided p-type materials alternate with a thermoelectric generator 82 comprising double-sided n-type materials. P-type and n-type thermoelectric generators can be arranged in any desired format. In some embodiments, p-type and n-type thermoelectric generators are arranged to balance resistance and/or thermoelectric performance of the p-type and n-type materials. FIG. 9, for example, illustrates an embodiment where a thermoelectric generator 91 comprising double-sided p-type materials is in electrical communication with two thermoelectric generators 92, 93 comprising double-sided n-type materials.

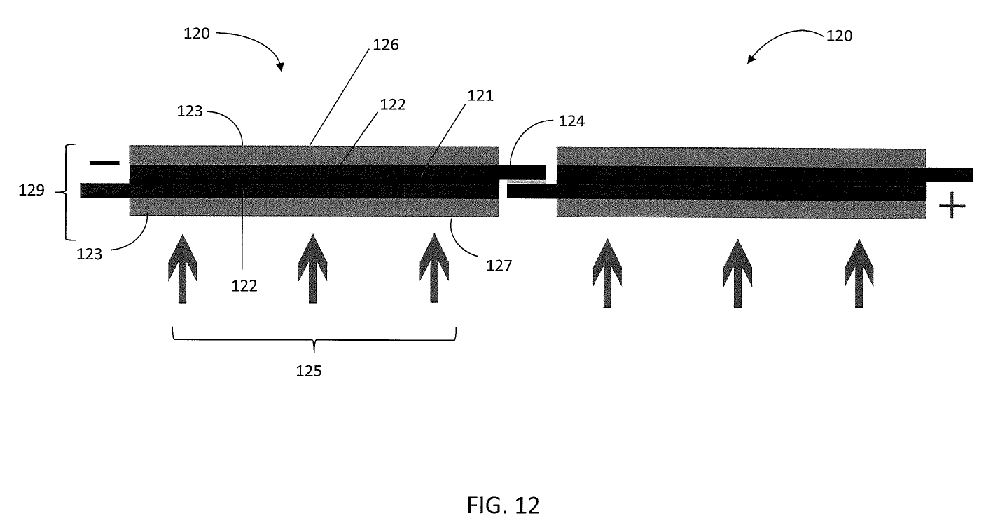

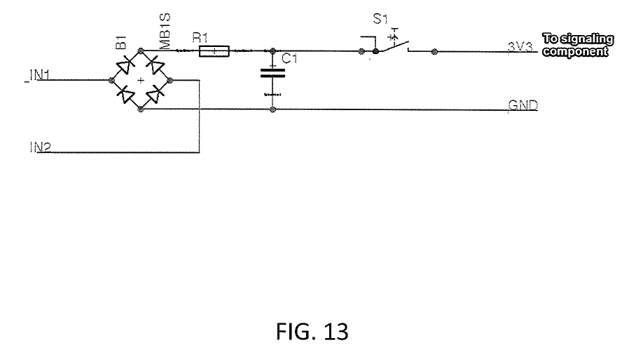

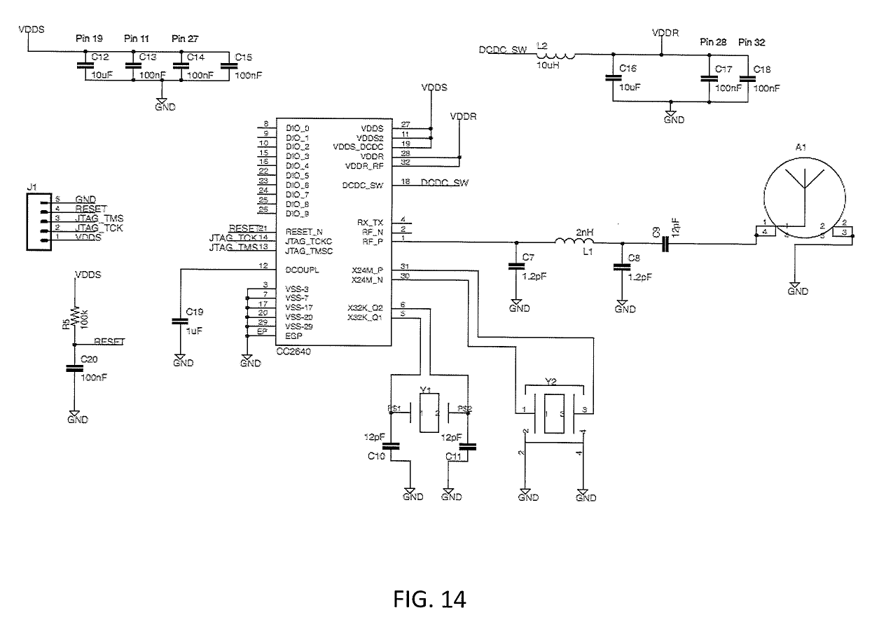

FIG. 12 illustrates a cross-sectional view of another embodiment of thermoelectric generators. In the embodiment of FIG. 12, the thermoelectric generators 120 comprise a p-type material or n-type material layer 121 contacting an electrically conductive layer 122. An outer electrically insulating layer 123 surrounds or encapsulates the p-type or n-type layer 121 and associated electrically conductive layer 122. Traces 124 can be employed to electrically connect individual thermoelectric generators 120 in series or parallel. In some embodiments, thickness of an individual thermoelectric generator 120 can generally range from 10 μm to 500 μm or 10 μm to 100 μm. Thickness of the thermoelectric generator 120 can be governed by several considerations including, but not limited to, the materials employed in the various layers 121-123 and environmental conditions in which the thermoelectric generator 120 is intended to operate. As illustrated in FIG. 12, a thermal gradient and/or heat flow 125 passes through the thickness 129 of the thermoelectric generator 120. This is in contrast to the thermoelectric generators of FIGS. 6-9 where thermal gradients are established along a length or width of the thermoelectric generator. In response to thermal gradients along the thickness, the thermoelectric generators 120 can exhibit a large surface area to thickness ratio. For example, a thermoelectric generator 120 can have a ratio of facial surface area to thickness ranging from 100 to 10,000. Facial surface area refers to the surface area of the top face 126 or bottom face 127 of the thermoelectric generator 120. By exhibit a high facial surface area to thickness ratio, the thermoelectric generators 120 can have plate-like or tile morphology. In some embodiments, a substrate can be tiled or paved with the thermoelectric generators 120 to provide a thermoelectric component of an electric generator described herein. The thermoelectric generators 120 can be tiled in any density not inconsistent with the objectives of the present invention. In some embodiments, a thermoelectric component comprises 100 to 1000 thermoelectric generators 120 per cm². Density of thermoelectric generators can be selected according to several considerations including, but not limited to, substrate size, desired amount of power generation and environmental conditions in which the thermoelectric generators are intended to operate.

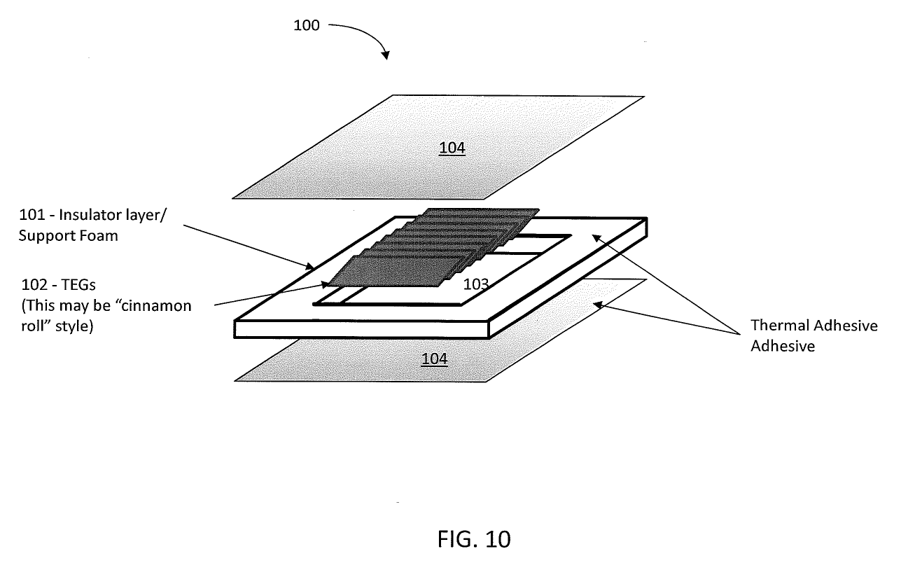

FIG. 10 illustrates an electric generator according to some embodiments described herein. The electric generator 100 comprises a support 101 and thermoelectric ribbon 102 positioned thereon. In the embodiment of FIG. 10, the support comprises a recess 103 in which the thermoelectric ribbon 102 is positioned. The thermoelectric ribbon 102 can have any construction and/or properties described in Section I hereinabove. In some embodiments, the thermoelectric ribbon 102 exhibits a folded and/or rolled configuration as illustrated in FIGS. 2-5. Moreover, the support 101 can be formed of any material not inconsistent with the objectives of the present invention. In some embodiments, the support 101 is formed of a flexible foam material. The support material can also exhibit a low thermal conductivity, generally less than 0.75 W/mK. For apparel applications, the support 101 can be formed of a material that is washable and non-permeable to water or other liquids. Protective layers 104 can be applied to encapsulate the thermoelectric ribbon 102 in the recess 103 of the support 101. The protective layers can be formed of a flexible material with high thermal conductivity, generally greater than 5 W/mK. The protective layers 102 can be secured to the support 101 via adhesive.

In some embodiments, the electric generator can further comprise a triboelectric component. The triboelectric component can include an electron donor layer in reversible contact with an electron acceptor layer. The electron donor layer and electron acceptor layer can exhibit a variety of contact modes. The electron donor and electron acceptor layers, for example, can be in a vertical contact separation mode. Alternatively, the electron donor and electron acceptor layers can have a lateral sliding mode arrangement. The electron donor layer and electron acceptor layer can be formed of any materials not inconsistent with triboelectric characteristics. In some embodiments, the electron donor layer and electron acceptor layer are formed of differing polymeric materials. For example, the electron donor layer and electron acceptor layer can be independently selected from the group of polymeric materials consisting of polyvinylidene fluoride (PVDF), polyvinyl fluoride (PVF), polyvinylidene fluoride-trifluoroethylene (PVDF-TrFE), polyvinylidene fluoride-tetrafluoroethylene (PVDF-TFE), polytetrafluoroethylene (PTFE), fluorinated ethylene propylene, polyethylene, polypropylene, elastomer and cellulose. One or more layers of metal or alloy may also be employed in the triboelectric component to facilitate or enhance the exchange of charge. In some embodiments, the triboelectric component can also exhibit piezoelectric behavior when piezoelectric polymeric materials are incorporated into the component construction.

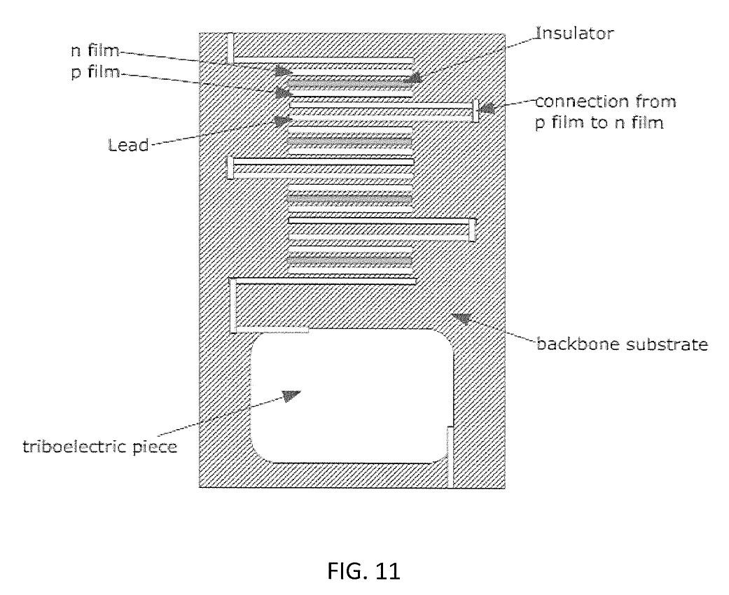

The triboelectric component can be in electrical communication with the thermoelectric component in any desired configuration. In some embodiments, the triboelectric component is connected in series with the thermoelectric component. In other embodiments, the triboelectric component is connected in parallel with the thermoelectric component. Further, the thermoelectric component is not limited to multilayer ribbon architectures described herein. Thermoelectric apparatus of various constructions can be paired with triboelectric component(s). For example, thermoelectric architectures described in PCT Patent Application PCT/US2011/056740 and/or PCT Patent Application PCT/US2014/027486 can be combined with triboelectric components in an electric generator. FIG. 11 illustrates an electric generator comprising a thermoelectric component and triboelectric component according to some embodiments described herein.

### III. Monitoring Systems

Electronic systems for incorporation into articles such as apparel and/or equipment are also described herein, which employ one or more energy harvesting mechanisms for powering various electronic functionalities. As described herein, such electronic functionalities include monitoring various aspects of wear and/or use of the apparel or equipment. In one aspect, a system for monitoring apparel/equipment wear or use comprises an energy harvesting component and a signaling component partially or fully powered by electrical energy generated by the energy harvesting component from wear or use of the apparel/equipment. In some embodiments, the monitoring system further comprises an energy storage component for storing electrical energy generated by the energy harvesting component. In such embodiments, the energy storage component can participate in powering of the signaling component.

Turning now to specific components, the energy harvesting component can comprise one or more apparatus operable to generate electrical energy from wear or use of the apparel or equipment. For example, the energy harvesting component can employ thermoelectric apparatus, piezoelectric apparatus, pyroelectric apparatus and/or triboelectric apparatus for generation of electrical energy. In some embodiments, apparatus of the energy harvesting component include various combinations of thermoelectric, piezoelectric, pyroelectric and/or triboelectric constructs. Apparatus of the energy harvesting component, in some embodiments, have construction and properties described in Sections I and II hereinabove. In other embodiments, apparatus of the energy harvesting component have construction and properties described in U.S. patent application Ser. No. 13/880,268 (Publication Number US 2013-0312806) and/or U.S. patent application Ser. No. 14/776,150 (Publication Number 2016-0035956), each of which is incorporated herein by reference in its entirety.

In some embodiments, an energy harvesting component comprises a triboelectric apparatus alone or in combination with thermoelectric, piezoelectric and/or pyroelectric apparatus. Triboelectric apparatus can include an electron donor layer in reversible contact with an electron acceptor layer. The electron donor layer and electron acceptor layer can exhibit a variety of contact modes. The electron donor and electron acceptor layers, for example, can be in a vertical contact separation mode. Alternatively, the electron donor and electron acceptor layers can have a lateral sliding mode arrangement. The electron donor layer and electron acceptor layer can be formed of any materials not inconsistent with triboelectric characteristics. In some embodiments, the electron donor layer and electron acceptor layer are formed of differing polymeric materials. For example, the electron donor layer and electron acceptor layer can be independently selected from the group of polymeric materials consisting of polyvinylidene fluoride (PVDF), polyvinyl fluoride (PVF), polyvinylidene fluoride-trifluoroethylene (PVDF-TrFE), polyvinylidene fluoride-tetrafluoroethylene (PVDF-TFE), polytetrafluoroethylene (PTFE), fluorinated ethylene propylene, polyethylene, polypropylene, elastomer and cellulose. One or more layers of metal or alloy may also be employed in the triboelectric component to facilitate or enhance the exchange of charge. In some embodiments, the triboelectric component can also exhibit piezoelectric behavior when piezoelectric polymeric materials are incorporated into the component construction.

By employing the foregoing constructions, the energy harvesting component is operable to generate electrical energy in response to the apparel or equipment being worn or used. Thermal gradients induced by temperature differences in the wearer and ambient environment may permit energy generation with thermoelectric apparatus. Moreover, heat from the wearer and/or environment can lead to electrical energy production by pyroelectric apparatus. Various mechanical stresses applied to the apparel during wear can be captured by piezoelectric and/or triboelectric apparatus for electrical energy production. Electrical energy generated by the energy harvesting component can be used to partially or fully power a signaling component. In response to receiving power generated by the energy harvesting component, the signaling component provides a signal conveying one or more types of information regarding the apparel. For example, the signaling component can provide a signal indicating the apparel is being worn or used. One more signals may also indicate time period(s) and/or activity levels of the wearer over which the apparel is worn or used.

The signaling component can also be combined with other sensory equipment or apparatus to increase information regarding the apparel. In some embodiments, the signaling component can be paired with a global positioning system such that a physical location can be assigned to the apparel when the signal is generated from use or wear of the apparel. The signal may also include information regarding the temperature of the apparel. For example, the signaling component may be programed to provide various signals depending on the amount and/or rate of energy received from the energy harvesting unit. Energy levels received from the energy harvesting unit can be calibrated to temperature or thermal gradients experienced by the energy harvesting unit, which can be correlated to temperature of the apparel, wearer and/or ambient environment. In some embodiments, this temperature information can be used to determine if an individual is in danger of overheating, heatstroke or hypothermia. In embodiments employing piezoelectric and/or triboelectric energy harvesting apparatus, energy levels received from the energy harvesting unit can be calibrated to movement and/or stresses experienced by the apparel. Such calibration can be correlated to physical activity of an individual wearing the apparel.

Signals provided by the signaling component can be in the form of radio waves or other electromagnetic radiation, including ultraviolet, visible and/or infrared radiation. In some embodiments, the radio waves can be in the Ultra High Frequency (UHF) band, generally having a frequency of 300 MHz to 3 GHz. For example, BLUETOOTH® or BLUETOOTH® low energy platforms and associated apparatus can be used as signaling components, in some embodiments. Signal generated by the signaling component can be transmitted to one or more electronic devices. Signal can be transmitted to any electronic device not inconsistent with the objectives of the present invention. Suitable electronic devices include, but are not limited to, cellular phones, smart phones, tablets and computers. The electronic device can be used to display and/or store information associated with or derived from the signal. Such information can be used to monitor wear and/or use of the clothing. Moreover, the information can monitor activity, health and/or location of an individual wearing or using the apparel. In some embodiments, the signal and information associated therewith can be paired with one or more applications on the electronic device. For example, signal indicating the apparel has been worn can be processed by an application on a smart phone, tablet or computer. An individual can obtain or accumulate reward points or gain access to various promotions by the apparel company based on wear or use of apparel as compiled by the application in conjunction with the monitoring system. Signal corresponding to physical activity or physical condition of an individual wearing the apparel can likewise be paired with a smart phone or tablet application for tracking physical activity and/or heath. Such data can also be paired with a reward system from the apparel company or third party including, employers, health care providers and/or health insurers.

In further embodiments, the signaling component can provide an audible signal and/or visual signal. A speaker and/or light emitting diode, for example, can signal when certain event criteria have been met. In some embodiments, an audible and/or visual signal from the signaling component can indicate dangerous body temperature of an individual wearing the apparel. In other embodiments, an audible and/or visual signal from the signaling component can indicate level(s) of physical activity have been achieved. As described above, the audible or visual signal can be tied to energy levels received from the energy harvesting component, which can be correlated to temperature of the apparel, energy harvesting component and/or ambient environment. Audible and/or visual signals can be combined with radio wave signals for communication with electronic apparatus.

In some embodiments, the monitoring system further comprises an energy storage component for storing electrical energy generated by the energy harvesting component. The energy storage component can subsequently power the signaling component upon receipt of a predetermined amount of electrical energy from the energy harvesting component. The predetermined amount of electrical energy can be set to correspond to a variety of events. For example, the predetermined amount can be set to a minimum duration or specific duration of wear or use of the apparel. In some embodiments, calibration charts can be developed to determine time periods required to generate predetermined amounts of electrical energy based on construction of the energy harvesting component and/or conditions in which the apparel is worn or used. Calibration charts can cover a variety of energy harvesting component constructions and wear or use conditions experienced by the apparel. Accordingly, the energy storage component can be constructed in accordance with one or more calibration charts to power the signaling device after certain temporal and/or environmental conditions have been met relating to wear or use of the apparel.

The energy storage component can have any construction and/or properties not inconsistent with the objectives of the present invention. In some embodiments, the energy storage component comprises one or more capacitors. The one or more capacitors can discharge to power the signaling component for indicating wear or use of the apparel. Capacitor discharge can occur when sufficient charge or electrical energy is stored to power the signaling component. In some embodiments, size and properties of the capacitor(s) can be selected according to calibration charts or other data such that discharge of the capacitor(s) corresponds to temporal and/or environmental condition(s) being met by wear or use of the apparel. Discharge of the capacitor(s) can reset the apparel monitoring system, thereby permitting additional signals to be generated as wear or use conditions are continued to be met over time.

In some embodiments, the energy storage component is used with an additional energy component to power the signaling component. The energy storage component, in some embodiments, can be used with one or more batteries to power the signaling component. For example, discharge of capacitor(s) can be used to signal or induce release of current from one or more batteries. This release of current can power the signaling component. FIGS. 14 and 15 are circuit diagrams for non-limiting embodiments of apparel monitoring systems according to some embodiments.

As described herein, the energy harvesting component partially or fully powers the signaling component. Alternatively, the energy harvesting component is not required to partially or fully power the signaling component. The energy harvesting component can reside in the power chain to the signaling component, but electrical energy produced by the energy harvesting component does not directly power the signaling component. For example, electrical energy generated by the energy harvesting component can be employed to initiate release of electrical energy from another component of the apparel monitoring system for powering the signaling component. In some embodiments, electrical energy produced by the energy harvesting component initiates or is in a process stream inducing release of electrical energy from one or more batteries.

Monitoring systems described herein can be coupled or applied to any type of apparel. Monitoring systems can be applied to shirts, pants, shorts, dresses, skirts, suits, underwear, socks, hats, helmets, shoes, boots, ties, jackets, lanyard, identification badge, keychain, athletic apparel, athletic equipment and personal protective equipment, such as equipment worn by the armed forces, police, fire fighters and/or construction workers. Moreover, components of the monitoring system can be positioned in a housing. The housing can be flexible and fabric-like for blending with apparel articles. The housing can permit the monitoring system to be washed and dried with the clothing, thereby obviating separate care requirements by the consumer.

In a further aspect, methods of monitoring use or wear of apparel are provided. In some embodiments, a method of monitoring use or wear of apparel comprises coupling a monitoring system to the apparel, the monitoring system comprising an energy harvesting component and a signaling component. Electrical energy is generated with the energy harvesting unit from wear or use of the apparel. The electrical energy is employed to partially or fully power the signaling component to emit a signal providing information regarding wear or use of the apparel. In some embodiments, the monitoring system further comprises an energy storage component for storing electrical energy generated by the energy harvesting component. Information regarding use or wear of the apparel, in some embodiments, comprises the time period in which the apparel is worn or the location of the apparel in a global positioning system. Information can also include temperature of the apparel and other types of information described above. The signal and associated information can be transmitted to and collected by one or more electronic devices as described hereinabove. Moreover, information processed by the electronic device can be used for various reward and promotional programs discussed hereinabove.

The foregoing principles of systems and methods for apparel monitoring can be employed in other applications, including asset or inventory management as well as monitoring use of equipment including, but not limited to, heavy equipment employed in the construction industry. For asset or inventory management, a system comprising an energy harvesting component and a signaling component partially or fully powered by electrical energy generated by the energy harvesting component can be applied or coupled to the asset or inventory. In some embodiments, for example, vibrations, mechanical stresses and/or temperature fluctuations associated with movement of the assets and/or inventory provide sufficient energy for collection by the energy harvesting component. Information regarding location and/or movement of the asset or inventory can be provided by the signaling component in conjunction with a component for receiving the signal. Receiving component(s) can comprise any suitable electrical device for receiving signals from the signaling component. In some embodiments, electronic devices of the signaling component comprise tablets and/or computers. The receiving component can record the signal and/or pass the signal on to one or more other devices for storage. The receiving component, for example, can pass the signal and associated information onto a database for access by a user of the system. In some embodiments, the database can be part of a cloud or remote storage location. Multiple receiving components can be part of the system for monitoring location and/or movement of the asset or inventory. Multiple receiving components can be stationary and/or mobile. In some embodiments, the inventory or assets are mobile, such as livestock, pets, cars, trucks, construction equipment and/or trains. In such embodiments, mobile receiving components can be employed for receiving signals from the signaling component. For instance, receiving components can be coupled to drones or other mobile devices for traveling various distances to receive signals from signaling components coupled to the mobile inventory. Accordingly, the asset or inventory can be located if missing or outside region(s) covered by stationary signal receiving components.

In some embodiments, monitoring systems coupled to the asset or inventory can be employed with apparel monitoring systems described herein. In this way, personnel in the vicinity of the inventory and/or asset can be readily identified. This can assist in full control and information regarding tracking and management of assets and/or inventory.

As described herein, a system comprising an energy harvesting component and signaling component can be coupled to equipment, including heavy equipment used in construction. Vibrations, mechanical stresses and/or temperature fluctuations associated with use of the heavy equipment can be harvested by the energy harvesting component for powering the signaling component. The signaling component can be employed to monitor usage and/or operating conditions of the heavy equipment. Such information can be stored and used for scheduling maintenance and management of the heavy equipment. Moreover, the monitoring system can provide location information of the heavy equipment according to principles described herein. In some embodiments, location information of the heavy equipment is relative to another object or person. In some embodiments, information regarding the relative location of the heavy equipment can be used in safety systems to prevent accident or injury. The signaling information provided by the monitoring system, for example, can be used to alert a user of the heavy equipment of being too close to an object or person. The warning can come in any format including visual, auditory and/or tactile. In some embodiments, internal safety systems of the heavy equipment may be alerted by the monitoring system, thereby enabling automatic shut-off or other safety mechanism. Monitoring systems coupled to heavy equipment can be employed with apparel monitoring systems described herein. In such embodiments, personnel in the vicinity of the inventory and/or asset can be readily identified. The personnel and/or heavy equipment can be alerted if the location of the personnel and heavy equipment fall within a separation distance threshold.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A system for monitoring apparel wear or use comprising:
an energy harvesting component; and
a signaling component partially or fully powered by electrical energy generated by the energy harvesting component from wear or use of the apparel.

2. The system of claim 1 further comprising an energy storage component for storing the electrical energy generated by the energy harvesting component.

3. The system of claim 2, wherein the energy storage component powers the signaling component upon receipt of a predetermined amount of electrical energy from the energy harvesting component.

4. The system of claim 3, wherein the predetermined amount of electrical energy corresponds to a period of time or user activity level over which the apparel is used or worn.

5. The system of claim 1, wherein the energy harvesting component comprises a thermoelectric apparatus.

6. The system of claim 5, wherein the thermoelectric apparatus comprises thermoelectric fabric.

7. The system of claim 1, wherein the energy harvesting component comprises piezoelectric apparatus.

8. The system of claim 1, wherein the energy harvesting component comprises a pyroelectric apparatus.

9. The system of claim 1, wherein the energy harvesting component comprises a triboelectric apparatus.

10. The system of claim 1, wherein the energy harvesting component comprises two or more apparatus selected from the group consisting of thermoelectric apparatus, piezoelectric apparatus, pyroelectric apparatus and triboelectric apparatus.

11. The system of claim 2, wherein the energy storage component comprises one or more capacitors.

12. The system of claim 1, wherein the signaling component employs electromagnetic radiation for communication with one or more electronic devices external to the system.

13. The system of claim 12, wherein the electronic device is a cellular phone, computer, tablet or electronic receiving system.

14. The system of claim 12, wherein the electronic device includes a global positioning system.

15. The system of claim 12, wherein the electromagnetic radiation comprises radio waves having frequency of 300 MHz to 3 GHz.

16. The system of claim 1 further comprising a housing in which the energy harvesting component, energy storage component and signaling component are positioned.

17. The system of claim 16, wherein the housing is flexible.

18. An apparel composition comprising:
an article of apparel; and
a system coupled to the article of apparel, the system comprising an energy harvesting component and a signaling component partially or fully powered by electrical energy generated by the energy harvesting component from wear or use of the apparel.

19. The apparel composition of claim 18, wherein the system further comprises an energy storage component for storing the electrical energy generated by the energy harvesting component.

20. The apparel composition of claim 19, wherein the energy storage component powers the signaling component upon receipt of a predetermined amount of electrical energy from the energy harvesting component.

21. The apparel composition of claim 20, wherein the predetermined amount of electrical energy corresponds to a period of time or user activity level over which the apparel is used or worn.

22. The apparel composition of claim 18, wherein the energy harvesting component comprises one or more apparatus selected from the group consisting of thermoelectric apparatus, piezoelectric apparatus, pyroelectric apparatus and triboelectric apparatus.

23. The apparel composition of claim 18, wherein the article of apparel is selected from the group consisting of a shirt, pants, shorts, dress, skirt, suit, underwear, sock, hat, helmet, shoe, boot, tie, jacket, lanyard, keychain, identification badge, athletic apparel and personal protective equipment.

24-75. (canceled)
